(19) United States
(12) Patent Application Publication
(10) Pub. No.: US 2013/0140341 A1
(43) Pub. Date: Jun. 6, 2013

(54) LINEAR STAPLED HEMORRHOIDECTOMY SYSTEMS AND METHODS

(71) Applicant: Darin M. Minkin, Chesterfield, MO (US)

(72) Inventor: Darin M. Minkin, Chesterfield, MO (US)

(21) Appl. No.: 13/310,053

(22) Filed: Dec. 2, 2011

(51) Int. Cl. A61B 17/068

(52) U.S. Cl. 227/176.1

(57) ABSTRACT

A hemorrhoidectomy tool is provided having a first arm comprising a first handle and a first linear jaw and a second arm pivotally connected to the first arm in a scissor-like manner. The second arm comprises a second handle and a second linear jaw. The handles can be opened to place the jaws in an Opened position whereafter at least one hemorrhoid bundle can be positioned between the jaws. Subsequently, the jaws can be closed to clasp the hemorrhoid bundle(s) therebetween. The tool additionally includes a linear staple cartridge comprising a plurality of staples. The tool is structured and operable to dispense the staples when the jaws are closed such that the staples pierce the clasped flesh of the hemorrhoid bundle(s) to form a linear suture along the base of the hemorrhoid bundle(s). Subsequently, the hemorrhoid bundle(s) can be excised and the tool removed leaving behind a linear sutured wound.

### FIELD

The present teachings relate to the substantially simultaneous excision and suturing of internal and external hemorrhoids utilizing a linear stapling hemorrhoidectomy tool.

### BACKGROUND

The statements in this section merely provide background information related to the present disclosure and may not constitute prior art.

Operative hemorrhoidectomies are generally performed utilizing a well-known “cut and suture” technique where a hemorrhoid bundle is surgically excised and the resulting wound is closed using standard suturing techniques. Cutting and suturing is standard practice for both the proctoplasty and the internal and external hemorrhoidectomy portion of the procedure. Removing hemorrhoids using the “cut and suture” technique can be a lengthy procedure and can incur significant bleeding. Furthermore, such known hemorrhoidectomy procedures are technically complicated and very difficult to perform on the morbidly obese who are often plagued by hemorrhoids requiring operation.

In some instances, circular staplers have been employed to close the resulting wound of internal hemorrhoids. However, such staplers are technically complicated and are very difficult to use and often ineffective in the morbidly obese. Moreover, these devices can only be used for internal hemorrhoids and they deploy a ring of hundreds of metal staples that permanently remain within the patient's rectum.

### SUMMARY

In various embodiments, the present disclosure provides a hemorrhoidectomy tool having a first arm comprising a first handle and a first linear jaw and a second arm pivotally connected to the first arm in a scissor-like manner. The second arm comprises a second handle and a second linear jaw. The handles can be opened to place the jaws in an Opened position whereafter at least one hemorrhoid bundle can be positioned between the jaws. Subsequently, the jaws can be closed to clasp the hemorrhoid bundle(s) therebetween. The tool additionally includes a linear staple cartridge comprising a plurality of staples. The tool is structured and operable to dispense the staples when the jaws are closed such that the staples pierce the clasped flesh of the hemorrhoid bundle(s) to form a linear suture along the base of the hemorrhoid bundle(s). Subsequently, the hemorrhoid bundle(s) can be excised and the tool removed leaving behind a linear sutured wound.

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is a side view of a hemorrhoidectomy system with a hemorrhoidectomy tool of the system disposed in a Closed Position, in accordance with various embodiments of the present disclosure.

FIG. 2 is a side view the hemorrhoidectomy system shown in FIG. 1 with the hemorrhoidectomy tool disposed in an Open Position, in accordance with various embodiments of the present disclosure.

FIG. 3 is an isometric view of a first jaw and a second jaw of the hemorrhoidectomy system shown in FIGS. 1 and 2, clasping a hemorrhoid bundle between the respective jaws, in accordance with various embodiments of the present disclosure.

FIG. 4 is a top view of a linear staple cartridge and a corresponding staple anvil of the hemorrhoidectomy system shown in FIGS. 1 and 2, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

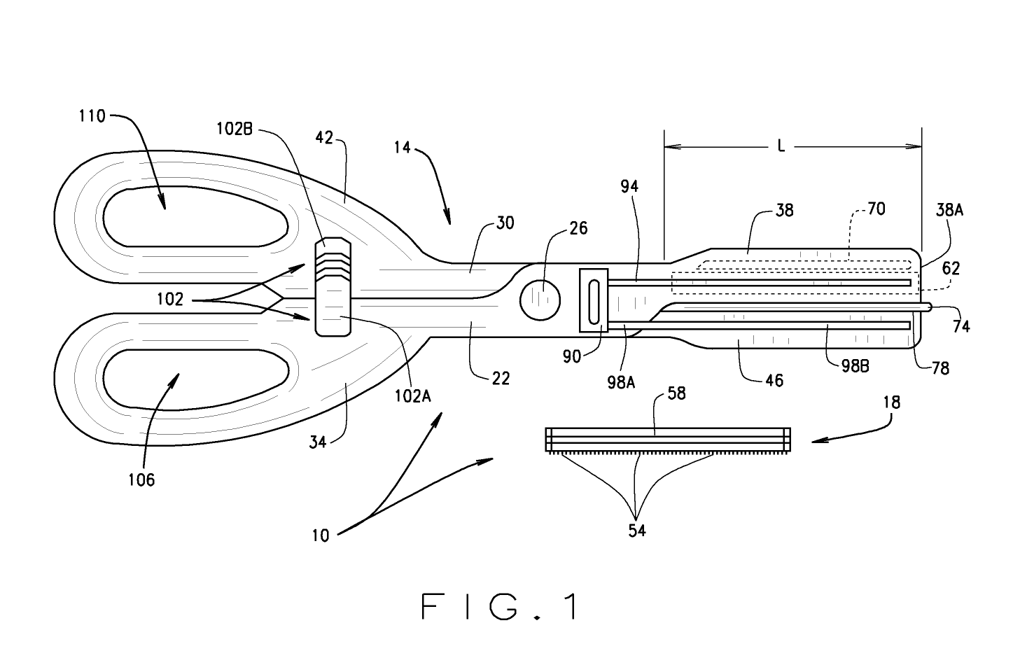

Referring to FIGS. 1, 2 and 3, the present disclosure provides a hemorrhoidectomy system 10 for substantially simultaneously excising and suturing in-line internal and external hemorrhoids. The system 10 includes a hemorrhoidectomy tool 14 and a linear staple cartridge 18 that is structured to be removably retained within the tool 14, as described further below. Generally, the system 10 is structured and operable to clasp one or more hemorrhoid bundles 50 and provide a linear suture along the base of the hemorrhoid bundles 50 utilizing a plurality of staggered staples 54, e.g., absorbable staples, dispensed from the cartridge 18 by the tool 14. As used herein, with regard the present description of the hemorrhoidectomy system 10, the terms ‘suture’, ‘linear suture’ and ‘suture line’ will be understood to mean a joining, i.e., a linear joining, of the lips or edges of a surgical wound made at the base of the hemorrhoid bundles using the staples 54. Subsequently, the hemorrhoid bundles can be excised just above the linear suture, i.e., the line of staples, resulting in an expedient hemorrhoidectomy absent the significant bleeding and the need for and difficulty of standard suturing.

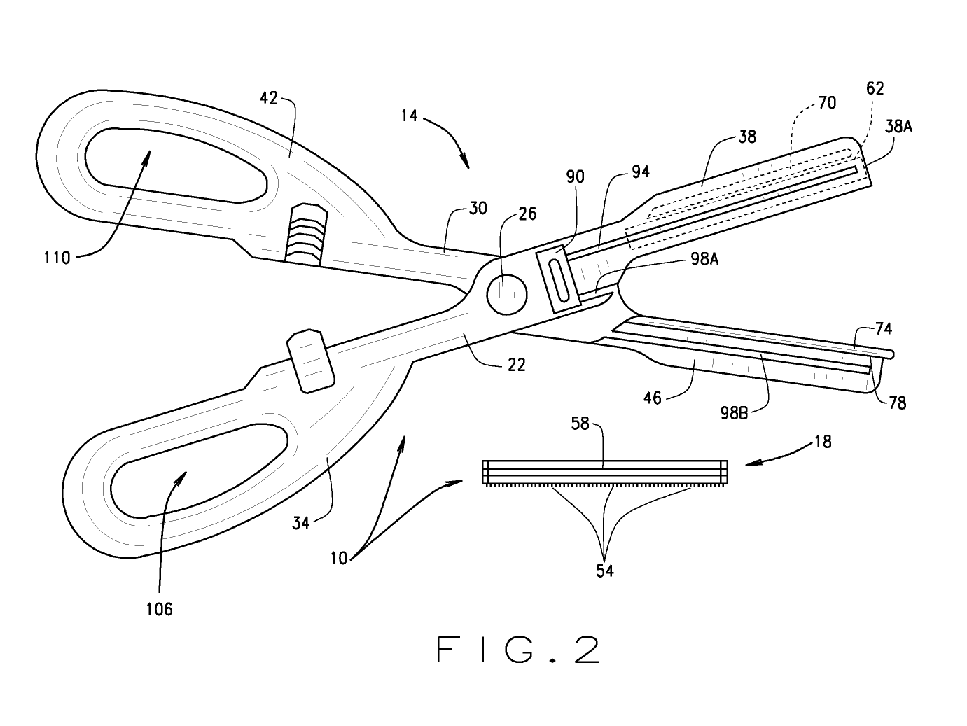

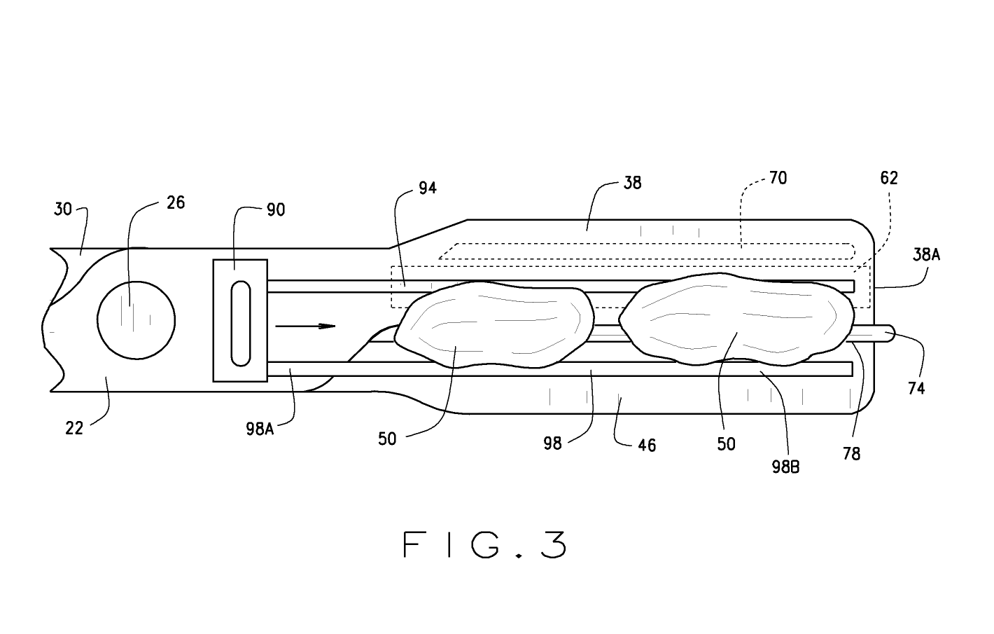

In various embodiments, the tool 14 includes a first arm 22 pivotally connected in a scissor-like manner, via a pivot connector 26, to a second arm 30. The pivot connector 26 can be any connector suitable for connecting the first and second arms 22 and 30 while allowing the first and second arms 22 and 30 to pivot in a scissor-like manner, e.g., a screw or rivet. The first arm 22 comprises a first handle 34 and a first linear jaw 38 extending from the first handle 34. Similarly, the second arm 30 comprises a second handle 42 and a second linear jaw 46 extending from the second handle 42. As described above, the first and second arms 22 and 30 are pivotally connected in a scissor-like manner, via the pivot connector 26. More specifically, the first and second arms 22 and 30 are pivotally connected such that positioning the first and second handles 34 and 42 in an ‘Opened Handle’ position (shown in FIG. 2) will correspondingly dispose the first and second linear jaws 38 and 46 in an ‘Opened Jaw’ position (shown in FIG. 2) such that at least one hemorrhoid bundle 50 (shown in FIG. 3) can be positioned between the first and second linear jaws 38 and 46. Furthermore, positioning the first and second handles 34 and 42 in a ‘Closed Handle’ position (shown in FIG. 1) will correspondingly place the first and second linear jaws 38 and 46 in a ‘Closed Jaw’ position such that the at least one hemorrhoid bundle 50 can be clasped therebetween (as illustrated in FIG. 3).

In various embodiments, the first and second jaws 38 and 46 are structured to have a length L suitable for clasping two or more hemorrhoid bundles 50 therebetween. For example, in various embodiments, the first and second jaws 38 and 46 are structured to have a length L that will allow at least one internal hemorrhoid bundle 50 and at least one external hemorrhoid bundle 50 to be simultaneously clasped between the first and second jaws 38 and 46. Accordingly, at least one internal hemorrhoid bundle 50 and the at least one external hemorrhoid bundle 50 to be simultaneously clasped therebetween and stapled to provide a linear suture, via the hemorrhoidectomy system 10, then subsequently excised, as described below.

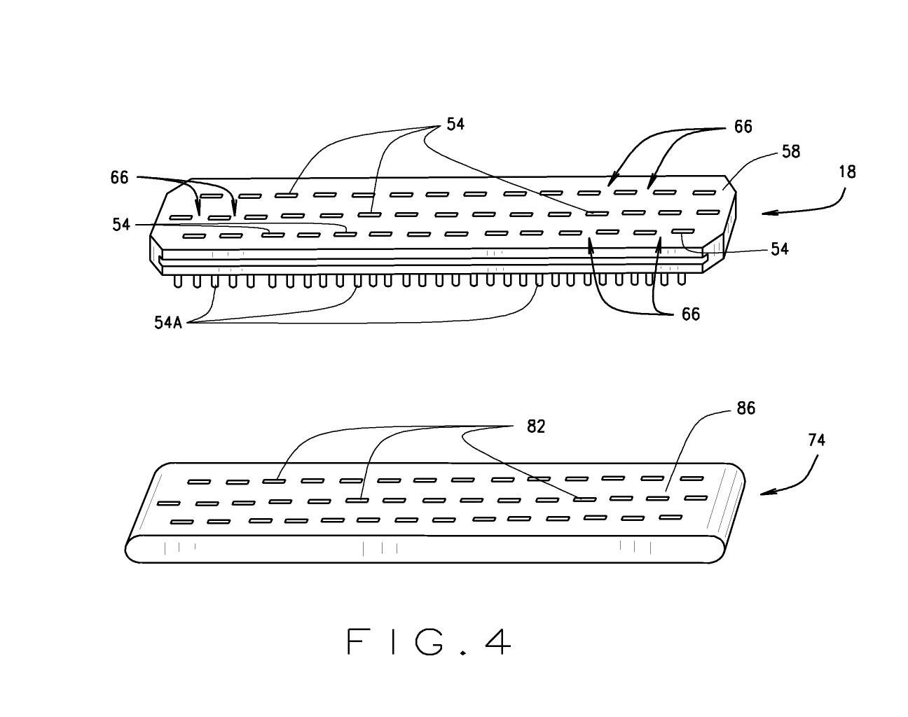

Referring now to FIGS. 1, 2, 3 and 4, the linear staple cartridge 18 comprises a plurality of staples 54 dispensibly retained within a spine 58. The staples 54 can be any type, composition, size and shape of staple suitable for the respective procedure. For example, in various embodiments, the staples 54 are fabricated of an absorbable material that will dissolve and be absorbed by the patient's body once the wound resulting from the respective procedure has completely healed. Additionally, in various embodiments, the staples 54 comprise a plurality of staggered rows of staples 54, wherein the staples 54 are disposed in the spine 58 in a staggered array. Particularly, the staples 54 in each row are disposed within the spine 58 having a space 66 between adjacent staples 54 within each respective row. More particularly, the staples 54 in adjacent rows are disposed within the spine 58 such that each sequential staple 54 of a particular row spans the space 66 between the two adjacent staples 54 of at least one adjacent row. Accordingly, the staples 54 are disposed within the spine 58 in a staggered array much like bricks are disposed within a wall in a staggered array. This staggered arrangement of the staples 54 provides a secure, well-sealed linear suture in the flesh at the base of the hemorrhoid bundle(s) 50, i.e., a secure, well-sealed linear suture along the resulting wound when the hemorrhoid bundle(s) 50 is/are excised.

The first jaw 38 comprises a linear staple cartridge retention fixture 54 that is structured to removably mate with and retain the linear staple cartridge 18 such that a first linear staple cartridge 18 can be easily and quickly inserted into the first jaw 38 and easily and quickly removed after use. The retention fixture 62 can be any fixture or structure suitable for removably mating with and retaining the staple cartridge 18. For example, it is envisioned that in various embodiments, the retention fixture 62 can comprise a reservoir having rails or tracks on opposing sidewalls that are structured to mate with channels formed along opposing sides of the staple cartridge spine 58. Accordingly, the staple cartridge 18 can be inserted through an opening in a distal end 38A of the first jaw 38 such that the staple cartridge channels slidingly mate with the retention fixture rails allowing the staple cartridge 18 to be slid into the reservoir, whereby the staple cartridge is retained and positioned for use, as described below. Subsequently, after the staples have been dispensed, as described below, the empty cartridge 18, i.e., the spine 58, can be removed by sliding the empty cartridge 18 back out the opening in the first jaw distal end 38A.

Additionally, the first jaw 38 includes a staple dispensing structure 70 that is operatively connected to the first handle 34. More specifically, the staple dispensing structure 70 is operatively connected to the first handle 34 such that when the first and second handles 34 and 42 are placed in the Closed position by physician, i.e., the physician squeezes the first and second handles 34 and 42 together, the staple dispensing structure 70 dispenses the staples 54 from the cartridge 18, i.e., the staple dispensing structure 70 pushes the staples 54 out of the spine 58. The staple dispensing structure 70 can be any structure, device or mechanism operable to dispense the staples 54 as described herein. For example, in various embodiments, the staple dispensing structure 70 can comprise a linear push bar operatively connected to the first handle 34 such that when the physician squeezes the first and second handles 34 and 42 together, the linear push bar 70 pushes the staples 54 out of the spine 58 causing the staples 54 to pierce hemorrhoid bundle(s) 50 clasped between the first and second linear jaws 38 and 46.

Moreover, the second jaw 46 includes a staple anvil 74 mounted to a face 78 of the second jaw 46. The staple anvil 74 comprises a plurality of staple crimping indents 82 that are arrayed on a face 86 of the anvil 74 to match/correspond with the array of the staples 54 within the spine 58. Additionally, the crimping indents 82 are structured and operable to receive and bend legs 54A of each staple 54 as each respective staple 54 is dispensed from staple cartridge 18 when the first and second jaws 38 and 46 are closed, clasping the internal and/or external hemorrhoid bundle(s) 50 between the first and second jaws 38 and 46.

More specifically, as the staples 54 are dispensed from the staple cartridge 18 by placing the first and second jaws 38 and 46 in the Closed position, the legs 54A of each staple 54 pierce the flesh along the bottom of the hemorrhoid bundle(s) clasped between the jaws 38 and 46. Once the staple legs 54A have pierced the flesh, the legs 54A are forced into a corresponding one of the crimping indents 82 of the anvil 74, via the staple dispensing structure 70. The contour of each indent 82 is such that as the staple legs 54A are forced into the indents 82, the legs 54A are bent or folded over, e.g., inward or outward, to secure the staples 54 within the flesh and provide a linear suture along the bottom of the hemorrhoid bundle(s) 50, as described further below. Subsequently, the hemorrhoid bundle(s) 50 can be excised and the tool 14 released, i.e., the first and second jaws 38 and 46 are placed in the Open position, leaving a well sutured wound, i.e., a wound where the lips of the wound are well sealed by the rows of staples 54, at the site where each excised hemorrhoid bundle 50 previously distended.

In various embodiments, the hemorrhoidectomy tool 14 includes a surgical cutting blade 90 slidingly engagable along the sides of the first and second jaws 38 and 46 such that once the hemorrhoid bundle(s) 50 have been clasped between the jaws 38 and 46 and stapled via dispensing of the staples 54, as described above, the blade 90 can be advanced along the sides of the jaws 38 and 46 to excise the hemorrhoid bundle(s) 50. For example, in various implementations, the tool 14 comprises a first blade guide channel 94 formed in the side of the first jaw 38 and a second blade guide channel 98 formed partially in the first jaw 38 and partially in the second jaw 46. That is, the second blade guide channel 98 has a first portion 98A formed in the side of the first jaw 38 that aligns with a second portion 98B formed in the side of the second jaw 46 when the first and second jaws 38 and 46 are placed in the Closed position, as shown in FIGS. 1 and 3. Accordingly, once the hemorrhoid bundle(s) 50 have been clasped between the jaws 38 and 46, and the staples 54 have formed a linear suture in the flesh along the bottom of the hemorrhoid bundle(s) 50, the first and second portions 98A and 98B of the second blade guide channel 98 will align allowing the cutting blade 90 to be advanced, via the guide channels 94 and 98, along the length of Closed first and second jaws 38 and 46, whereby the surgical cutting blade 90 will excise the hemorrhoid bundle(s) 50 above the linear suture.

Referring now to FIGS. 1 and 2, in various embodiments, the first handle 34 includes a first connector 102A of the locking mechanism 102, and the second handle 42 comprises a second connector 102B of the locking mechanism 102. The first and second connectors 102A and 102B are structured and operable to interlockingly mate with each other such that the first and second jaws 38 and 46 can be disengagingly locked in the Closed position, as illustrated in FIG. 1. Therefore, once the physician has clasped the hemorrhoid bundle(s) 50 and stapled the flesh at the base of each hemorrhoid bundle 50, the first and second handles 34 and 42 can be temporarily locked together in the Closed position, thereby maintaining the first and second jaws 38 and 46 in the Closed position. Subsequently, the physician can excise the hemorrhoid bundle(s) 50, via the surgical cutting blade 90 or any other suitable surgical cutting instrument, e.g., a surgical scalpel or scissor. After the hemorrhoid bundle(s) 50 has/have been excised, the first and second connectors 102A and 102B of the locking mechanism 102 can be disengaged to transition the jaws 38 and 46 to the Open position, whereafter the hemorrhoidectomy tool 14 can be removed.

It is envisioned that the first and second handles 34 and 42 can have any shape, size and structure suitable for easily and conveniently moving the handles 34 and 42, and hence, the jaws 38 and 46, between the respective Open and Closed positions. For example, in various embodiments, the first handle 34 comprises a first finger retention ring or loop 106 that is structured and operable to receive and retain the a thumb or one or more figures of the surgeon. Similarly, the second handle 42 comprises a second finger retention ring or loop 110 that is structured and operable to receive and retain the corresponding thumb or one or more figures of the surgeon. Hence, the first and second handles 34 and 42 are structured as scissor-like handles that are ergonomically comfortable and easy for the surgeon to manipulate in a scissor-like manner in order to operate the hemorrhoidectomy tool 14 using only one hand, as described herein.

Referring now to FIGS. 1, 2, 3 and 4, to utilize the hemorrhoidectomy system 10 to excise or remove one or more hemorrhoid bundles 50, e.g., an internal and an external hemorrhoid bundle 50, and substantially simultaneously suture the resulting wound, i.e., join together the lips of the wound, the surgeon (or surgical assistant) first inserts a linear staple cartridge 18 into the first jaw 38. Next, the surgeon exposes the hemorrhoid bundles 50 to be excised. The surgeon then opens the jaws 38 and 46 of the hemorrhoidectomy tool 14 by placing the handles 34 and 42 in the Open position and positions the one or more hemorrhoid bundles 50 to be excised between the jaws 38 and 46. Specifically, the surgeon places the jaws 38 and 46 on opposing sides of the hemorrhoid bundle(s) 50 at the base of the respective hemorrhoid bundle(s) 50. Next, the surgeon closes the handles 34 and 42, i.e., squeezes the handles 34 and 42 together, thereby closing the jaws 38 and 46 and clasping the hemorrhoid bundle(s) 50 therebetween. In various embodiments, as the handles 34 and 42 are squeezed together, the first and second connectors 102A and 102B of the locking mechanism 102 interlockingly engage.

The surgeon continues to squeeze the handles 34 and 42 closer together, thereby linearly flattening the base(s) hemorrhoid bundle(s) 50 at the location where the respective hemorrhoid bundle(s) are tightly clasped, or pinched, between the linear jaws 38 and 46. Moreover, as the handles 34 and 42 are squeezed closer together the staple dispensing structure 70 begins to dispense the linear rows of staples 54, e.g., absorbable staples, from the spine 58 such that the staples begin to pierce the flesh at the base of the hemorrhoid bundle(s) 50. Continued squeezing of the handles 34 and 42 will cause the staple dispensing structure 70 to push the staples 54 through the flesh clasped between the jaws 38 and 46 forcing the tips of the staple legs 54A of each staple 54 into the corresponding crimping indents 82 of the staple anvil 74. Further squeezing of the handles 34 and 42 will cause the staple dispensing structure 70 to continue to apply force to the staples 54 such the staple legs 54A are folded or bent over, e.g., inward or outward, to secure the staples 54 within the flesh, thereby providing a well-sealed linear suture along the bottom of the hemorrhoid bundle(s) 50.

Once the linear suture is completed, i.e., the rows of staples 54 have been disposed at the base of the hemorrhoid bundle(s) 50, the surgeon can excise the hemorrhoid bundle(s) 50 using a suitable surgical instrument. For example, in various embodiments wherein the hemorrhoidectomy tool 14 includes the cutting blade 90, the surgeon can advance the cutting blade 90 along the sides of the closed jaws 38 and 46, via the first and second guide channels 94 and 98. As the cutting blade 90 is advanced along the sides of the jaws 38 and 46, the cutting blade 90 cuts through and excises the hemorrhoid bundle(s) 50. After the hemorrhoid bundle(s) 50 has/have been excised, the surgeon can open the handles 34 and 42, and subsequently the jaws 38 and 46, thereby releasing the sutured flesh, i.e., the stapled flesh, from between the jaws 38 and 46. Particularly, the hemorrhoidectomy tool 14 can be removed leaving behind the well-sealed linear sutured wound where the excised hemorrhoid bundle(s) 50 previously distended.

As described above, the hemorrhoidectomy system 10 is structured and operable to excise one or more hemorrhoid bundle(s) while substantially simultaneously linearly suturing the resulting wound. It is envisioned that the hemorrhoidectomy system 10 will greatly reduce the time consumed in performing hemorrhoidectomies by known systems and methods, greatly reduce the amount of blood loss, i.e., increase hemostasis, that commonly occurs when performing hemorrhoidectomies using known systems and methods, and greatly reduce the complexity and difficulty of performing hemorrhoidectomies using the known systems and methods, particularly in the morbidly obese.

The description herein is merely exemplary in nature and, thus, variations that do not depart from the gist of that which is described are intended to be within the scope of the teachings. Such variations are not to be regarded as a departure from the spirit and scope of the teachings.

What is claimed is:

1. A hemorrhoidectomy tool, said tool comprising:
a first arm comprising a first handle and a first linear jaw extending from the first handle; and
a second arm comprising a second handle and a second linear jaw extending from the second handle, the first and second arms pivotally connected in a scissor-like manner via a pivot connector such that positioning the first and second handles in an Opened Handle position will correspondingly dispose the first and second linear jaws in an Opened Jaw position such that at least one hemorrhoid bundle can be positioned between the first and second linear jaws, and positioning the first and second handles in a Closed Handle position will correspondingly place the first and second linear jaws in a Closed Jaw position such that the at least one hemorrhoid bundle can be clasped therebetween,
the first linear jaw comprising a cartridge retention fixture for removably retaining a linear staple cartridge comprising a plurality of absorbable staples, the first linear jaw structured and operable to dispense the staples from the staple cartridge such that legs of the staples pierce the at least one hemorrhoid bundle clasped between the first and second linear jaws when placed in the Closed Jaw position, and
the second linear jaw comprising a staple anvil structured to bend the legs of the staples such that the staples form a linear suture along the at least one hemorrhoid bundle.

2. The tool of claim 1, wherein:
the first handle comprises a first finger retention ring structured and operable to receive and retain one of a thumb and at least one figure of an operator during operation of the tool; and
the second handle comprises a second finger retention ring structured and operable to receive and retain one of a thumb and at least one figure of the operator during operation of the tool.

3. The tool of claim 1 further comprising a locking mechanism, wherein:
the first handle comprises a first connector of the locking mechanism; and
the second handle comprises a second connector of the locking mechanism, the first and second connectors structured and operable to mate such that the first and second jaws can be disengagingly locked in the Closed Jaw position.

4. The tool of claim 1 further comprising a cutting blade slidingly engagable with the first and second jaws such that the cutting blade can be advanced along the length of the first and second jaws when in the Closed Jaw position to excise the at least one hemorrhoid bundle above the linear suture formed by the absorbable staples.

5. The tool of claim 1, wherein the first jaw comprises a linear push bar structured and operable to dispense the staples from the cartridge such that the legs of the staples pierce the at least one hemorrhoid bundle clasped between the first and second linear jaws when placed in the Closed Jaw position.

6. A hemorrhoidectomy system for substantially simultaneously removing in-line internal and external hemorrhoids, said system comprising:
a hemorrhoidectomy tool comprising:
a first arm comprising a first handle and a first linear jaw extending from the first handle, the first linear jaw including a linear staple cartridge retention fixture; and
a second arm comprising a second handle and a second linear jaw extending from the second handle, the first and second arms pivotally connected in a scissor-like manner via a pivot connector such that positioning the first and second handles in an Opened Handle position will correspondingly dispose the first and second linear jaws in an Opened Jaw position such that at least one internal hemorrhoid bundle and at least one external hemorrhoid bundle can be substantially simultaneously positioned between the first and second linear jaws, and positioning the first and second handles in a Closed Handle position will correspondingly place the first and second linear jaws in a Closed Jaw position such that the internal and external hemorrhoid bundles can be clasped therebetween; and
a linear staple cartridge comprising a plurality of absorbable staples, the linear staple cartridge structured to removably mate with the linear cartridge retention fixture,
the first linear jaw structured and operable to dispense the staples from the staple cartridge such that legs of the staples pierce the internal and external hemorrhoid bundles clasped between the first and second linear jaws when placed in the Closed Jaw position, and
the second linear jaw comprising a staple anvil structured to bend the legs of the staples such that the staples form a linear suture along the internal and external hemorrhoid bundles.

7. The system of claim 6, wherein:
the first handle comprises a first finger retention ring structured and operable to receive and retain one of a thumb and at least one figure of an operator during operation of the tool; and
the second handle comprises a second finger retention ring structured and operable to receive and retain one of a thumb and at least one figure of the operator during operation of the tool.

8. The system of claim 6 further comprising a locking mechanism, wherein:
the first handle comprises a first connector of the locking mechanism; and
the second handle comprises a second connector of the locking mechanism, the first and second connectors structured and operable to mate such that the first and second jaws can be disengagingly locked in the Closed Jaw position.

9. The system of claim 6 further comprising a cutting blade slidingly engagable with the first and second jaws such that the cutting blade can be advanced along the length of the first and second jaws when in the Closed Jaw position to excise the internal and external hemorrhoid bundles above the linear suture formed by the absorbable staples.

10. The system of claim 6, wherein the first jaw comprises a linear push bar structured and operable to dispense the staples from the cartridge such that the legs of the staples pierce the internal and external hemorrhoid bundles clasped between the first and second linear jaws when placed in the Closed Jaw position.

11. The system of claim 6, wherein the linear staple cartridge comprises a plurality of staggered rows of staples.

12. A method for substantially simultaneously removing in-line internal and external hemorrhoids, said method comprising:
positioning a first handle of a first arm of a hemorrhoidectomy tool and a second handle of a second arm of the hemorrhoidectomy tool in an Open Handle position such that a first linear jaw extending from the first handle and a second linear jaw extending from the second handle are correspondingly disposed in an Open Jaw position, the first and second arms pivotally connected in a scissor-like manner via a pivot connector;
removably mating a linear staple cartridge comprising a plurality of absorbable staples with a linear cartridge retention fixture of the first linear jaw;
substantially simultaneously positioning at least one internal hemorrhoid bundle and at least one external hemorrhoid bundle between the first and second linear jaws when in the Open Jaw position;
clasping the internal and external hemorrhoid bundles between the first and second jaws, substantially simultaneously dispensing the staples from the staple cartridge, via a linear push bar of the first jaw, such that legs of the staples pierce the internal and external hemorrhoid bundles, and bending the legs of the staples, via an anvil of the second jaw, to form a linear suture along the internal and external hemorrhoid bundles by positioning the first and second handles in a Closed Handle position such that the first and second jaws are correspondingly placed in a Closed Jaw position wherein the internal and external hemorrhoid bundles are clasped therebetween and the staples are dispensed.

13. The method of claim 12, wherein clasping the internal and external hemorrhoid bundles between the first and second jaws comprises:
inserting one of a thumb and at least one figure of an operator into a first finger retention ring of the first handle;
inserting one of a thumb and at least one figure of the operator into a second finger retention ring of the second handle; and
squeezing the first and second retention rings, via the respective thumb and at least one finger of the operator, in a scissor-like manner to bring the first and second retention rings in close proximity to each other, thereby clasping the internal and external hemorrhoid bundles between the first and second jaws.

14. The method of claim 13, wherein clasping the internal and external hemorrhoid bundles between the first and second jaws further comprises squeezing the first and second retention rings in a scissor-like manner until a locking mechanism first connector of the first handle disengagingly mates with a second connector of the locking mechanism, the second handle comprising the second connector, such that the internal and external hemorrhoid bundles are fixedly clasped between the first and second jaws.

15. The method of claim 12 further comprising advancing a cutting blade, slidingly engagable with the first and second jaws, along the length of the first and second jaws when in the Closed Jaw position to excise the internal and external hemorrhoid bundles above the linear suture formed by the absorbable staples.

16. The method of claim 12, wherein dispensing the staples from the staple cartridge comprises dispensing a plurality of staggered rows of staples from the linear staple cartridge.
